(19) United States
(12) Patent Application Publication
(10) Pub. No.: US 2026/0260246 A1
(43) Pub. Date: Sep. 3, 2026

(54) SYSTEM(S) AND METHOD(S) FOR REGULATORY PRODUCT LIFECYCLE MANAGEMENT WITH REGULATORY INTELLIGENCE

(71) Applicant: M/S DDREG PHARMA PRIVATE LIMITED, Gurugram (IN)

(72) Inventors: Neeraj PANT, Gurugram (IN); Rahul ARYA, Gurugram (IN)

(21) Appl. No.: 18/845,755

(22) PCT Filed: Mar. 17, 2023
(86) PCT No.: PCT/IN2023/050259

§ 371 Date: Sep. 10, 2024

(30) Foreign Application Priority Data
Mar. 19, 2022 (IN) 202211015268

(51) Int. Cl. G06Q 30/018; G06F 21/62; G16H 70/40

(52) U.S. Cl. CPC G06Q 30/018; G06F 21/6218; G16H 70/40

(57) ABSTRACT

A system and a method for regulatory lifecycle management for a drug substance or drug product with dynamic integration of regulatory intelligence is disclosed. The system enables automation of project workflow by integrating project management, document management, and regulatory information management. In the project management, the system enables allocation of projects to different entities (resources). Further, the system allows tracking and monitoring of product registration in real-time including the product registration status worldwide.

Furthermore, the system maintains a complete audit trail of each of the projects created in the system. The system integrates the document management with the regulatory information management to provide access to data associated with regulatory authority requirements of different countries/regions.

### FIELD OF THE INVENTION

The present invention relates to systems and methods for Regulatory Product lifecycle management with dynamic integration of Regulatory intelligence and registration tracking.

### BACKGROUND

In the realm of pharmaceutical & biological product (referred together as ‘product’, hereinafter) access understanding of regulatory landscape with respect to country and/or region holds paramount importance. For gaining market authorization for product from regulatory authority of the country/region as well as for ensuring that the product remains compliant even when it is available on pharmacy shelves, the company or Marketing Authorization Holder (MAH) has to be proficient with current regulatory laws/regulations/guidelines/practices and has to remain updated all the time. This is regulatory intelligence and it has gained significant importance to ensure that the product complies with regulatory requirements for different countries and regions at each stage of product lifecycle, to ensure market access in key regions. The task of filing applications for product registrations and post approval lifecycle management becomes much challenging with dynamic nature of regulatory laws/regulations/guidelines/practices. Currently, various manual systems are employed for monitoring, gathering, and analyzing regulatory requirements that need to be implemented. However, with dynamic nature of regulatory laws/regulations/guidelines/practices together with the increasing complexity of products offerings access to regulatory intelligence at each stage of product lifecycle is the need of the hour. Further, Regulatory intelligence finds its extreme usage while putting together a submission package for a New Product/Generic approval or a variation. This leads to a cumbersome task for resources or entities involved in the project to access the regulatory requirements separately while working on various projects or submissions during each stage of product lifecycle. This also increases the overall time consumed for preparation of submission package. Therefore, there is a requirement of integrating regulatory intelligence with regulatory project lifecycle to provide access to regulatory information at each stage of submission package development.

### OBJECTS OF THE INVENTION

A general object of the present invention is to provide a system and method for pharmaceutical product regulatory lifecycle management with dynamic integration of regulatory intelligence during each stage of drug product development.

An object of present invention is to provide a system to track and monitor the products registration status worldwide.

An object of the present invention is to provide an automated platform for reviewing the technical documents pertaining to drug products or substances and other products that are to be registered with drug regulatory agency in a specific country.

An object of the present invention is to facilitate easier management of workflow pertaining to review, changes, updates to drug product dossier, and compilation in Common Technical Document (CTD) format.

An object of the present invention is to provide a system and method where regulatory projects can be internally tracked and monitored by the person having access to the system,

An object of the present invention is to provide dynamic access to regulatory requirements of 130+ countries on the same platform as document management and submission management.

An object of the present invention is to provide access to queries and experiences with country agencies so as to develop a robust dossier thereby avoiding major queries and leading to substantial saving of energy and time.

An object of the present invention is to provide a combination of project management with document management and regulatory information management system on a single platform.

An object of the present invention is to provide a project management system that allows project allocation to different resources/entities, multiple levels of project working, allows complete tracking and monitoring of projects on real time-basis, and maintains a complete audit trail.

An object of the present invention is to provide a document management system that allows working on project-related individual documents for reviewing, commenting, and authoring, allows the use of different document management applications, maintains complete document status and document audit trail, allows creating reports on the status of projects and documents, and allows creating a Common Technical Document (e.g. CTD) based database of working documents.

An object of the present invention is to provide regulatory information management that system that allows creating a repository of the regulatory information, integrating regulatory information with lifecycle management (project management and document management).

### SUMMARY

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present invention, a system and a method for regulatory lifecycle management with dynamic integration of regulatory intelligence is disclosed. The system may enable the automation of project workflow by integrating project management, document management, and regulatory information management. In the project management, the system may enable the allocation of projects to different resources or entities involved in the project, such as an administrator, an approver, a reviewer, and a doer. The system may allow different resources/entities to perform authorized actions on each of the projects. Further, the system may allow tracking and monitoring of projects in real-time. Furthermore, the system may maintain a complete audit trail of each of the projects created in the system. In the document management, the system may allow access to various documents associated with a project to different resources/entities.

Further, the system may allow each of the resources or entities involved in the project to perform various actions on each module of the dossier based on access rights assigned to each of the resources/entities of the project.

The system may integrate the document management with the regulatory information management to provide access to data associated with regulatory requirements of different countries/regions while performing various actions on individual documents for submission. In the regulatory information management, the system may be configured to collect, collate, analyze, and report data associated with regulatory information of different countries or regions. The system may be configured to manage a database of regulatory information which can be accessed by a resource or entity involved in the project to retrieve regulatory information relevant to performing an action on a document associated with a project or a submission.

Further, the system creates a repository of the data used for filing the dossier in different countries. It may allow each of the resources or entities involved in a project to review the earlier filed or approved dossiers in a particular country so as to capture the information based on the learning of review of past dossiers by a regulatory agency.

To further clarify the advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

FIG. 1 illustrates a schematic view of a system for regulatory lifecycle management with regulatory intelligence, according to an embodiment of the present invention.

FIG. 2 illustrates a block diagram of the system for regulatory lifecycle management with regulatory intelligence, according to an embodiment of the present invention.

FIG. 3A illustrates a flowchart depicting steps involved in the method for managing the regulatory lifecycle management with regulatory intelligence, according to an embodiment of the present invention.

FIG. 4A illustrates an exemplary flowchart depicting an operation of the system for managing the regulatory project lifecycle associated with a medicinal drug product, according to an embodiment of the present invention.

FIG. 4B illustrates a flowchart depicting data communication within the system for managing the regulatory project lifecycle with regulatory intelligence, according to an embodiment of the present invention.

FIG. 5A illustrates a flowchart depicting the creation and extraction of a project by implementing the system for managing the regulatory project lifecycle with regulatory intelligence, according to an embodiment of the present invention.

FIG. 5B illustrates a flowchart depicting the creation and extraction of submission by implementing the system for managing the regulatory project lifecycle with regulatory intelligence, according to an embodiment of the present invention.

FIG. 5C illustrates a flowchart depicting data uploading associated with a submission by implementing the system for managing the project lifecycle with regulatory intelligence, according to an embodiment of the present invention.

FIG. 5D illustrates a flowchart depicting data exporting associated with a submission or a project by implementing the system for managing the project lifecycle with regulatory intelligence, according to an embodiment of the present invention.

FIG. 6A illustrates a flowchart depicting an overview and working of a regulatory life cycle tracking operation, according to an embodiment of the present invention.

FIG. 6B illustrates a flowchart depicting the process flow for processing information on “Registration” status in the regulatory life cycle tracking operation, according to an embodiment of the present invention.

FIG. 6C illustrates a flowchart depicting the process flow for processing information on “Variation” status in the regulatory life cycle tracking operation, according to an embodiment of the present invention.

FIG. 6D illustrates a flowchart depicting the process flow for processing information on “Renewal” status in the regulatory life cycle tracking operation, according to an embodiment of the present invention.

FIG. 6E illustrates a flowchart depicting the report extraction process during the regulatory life cycle tracking operation, according to an embodiment of the present invention.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having the benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having an ordinary skill in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfill the requirements of uniqueness, utility, and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do NOT necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should NOT be necessarily taken as limiting factors to the attached claims. The attached claims and their legal equivalents can be realized in the context of embodiments other than the ones used as illustrative examples in the description below.

According to an aspect, the present invention elaborates upon a system for regulatory project lifecycle management of drug products with regulatory intelligence. The system comprises a processing unit comprising a processor coupled with a memory, wherein the memory stores one or more instructions executable by the processor. The processing unit is configured to receive a request pertaining to accessing the system from one or more registered entity through an electronic device, authenticate the entity, and upon authentication, parse the received request, wherein the authenticated entity is classified into one of the multiple groups, and identify a dataset associated with one or more projects from the parsed request based on access rights assigned to the entity for development of the corresponding project, and grant access to at least a portion of the identified dataset to the entity based on the classified group of the entity. The system allows the entity to perform one or more operations, through the electronic device, over the accessed portion of the dataset, wherein the one or more operations comprise any or a combination of creation, modification, deletion, reviewing, comment, approval, rejection, authoring, and uploading.

In an embodiment, a database associated with the system is integrated with drug regulatory authority guidelines and regulations associated with one or more jurisdictions. The system is configured to provide the entity with the drug regulatory authority guidelines and regulations of the one or more jurisdictions while performing the assigned actions on the dataset at each stage of the project development, and grant access and allow the entity to perform the one or more operations on the dataset at each stage of the project development based on the regulatory authority regulations of the corresponding jurisdiction. The regulatory project lifecycle management includes a filing, registration, renewal and any variation of already approved drug substances and/or drug products.

In an embodiment, the group of the entity comprises any of administrator, approver, reviewer, and doer. Further, the dataset includes any or a combination of a document, a folder, and a file.

In an embodiment, during registration, the system maps, through one or more tags, the entity with authentication data comprising any or a combination of a generic or proprietary name tag, a product tag, a company tag, and a performance tag, and correspondingly generates and stores a set of credentials in a database. Further, when the entity feeds the set of credentials into the system, the system authenticates the entity using the stored set of credentials and correspondingly identifies the access rights, and grants access to the authenticated entity. A generic or proprietary name tag includes the generic or chemical name of the drug, whereas the proprietary name includes the brand or trade name of the product.

In an embodiment, the system is configured to allow tracking and monitoring of the one or more projects and the corresponding dataset in real-time; and create one or more reports on the status of the one or more projects and the dataset associated with the corresponding project.

In an embodiment, the system is configured to facilitate the administrator to extract parameters of a designated project among the one or more projects, and correspondingly create and add one or more templates for a document associated with the designated project in a database, and allow the one or more registered entity to access the added template based on the requirement and the access rights assigned to the entity, to enable the preparation of the document and subsequently the dossier.

In an embodiment, the system enables the approver to create, edit, and delete the project, and create and upload a submission corresponding to a structure of the one or more templates. Further, the system is configured to dynamically provide the drug regulatory authority guidelines and regulations from the database to the approver while performing the actions.

In an embodiment, the reviewer accesses the system and approves or rejects the uploaded submission. Further, upon approval, the system uploads the corresponding data to the database.

In an embodiment, in case the reviewer rejects the submission, the system allows the doer to rectify errors in the corresponding data and subsequently allows the doer to upload the updated submission data for the review of the reviewer.

In an embodiment, the processing unit is associated with any of the electronic devices, a central server that is in communication with the electronic device, and a computing device that is in communication with the electronic device and/or the server.

According to another aspect, the present invention elaborates upon a method for project lifecycle management for a drug product or drug substance with regulatory intelligence. The method comprising the step of: receiving, by a processing unit, a request pertaining to accessing one or more projects from one or more registered entity through an electronic device; authenticating, by the processing unit, the entity, and upon authentication, parsing the received request, wherein the authenticated entity is classified into one of multiple groups; identifying, by the processing unit, a dataset associated with the one or more projects from the parsed request based on access rights assigned to the entity for development of the corresponding project; granting, by the processing unit, access to at least a portion of the identified dataset to the entity based on the classified group of the entity; and allowing, by the processing unit, the entity to perform one or more operations, through the electronic device, over the accessed portion of the dataset, wherein the one or more operations comprise any or a combination of creation, modification, deletion, reviewing, comment, approval, rejection, authoring, and uploading.

In an embodiment, a database in communication with the processing unit is integrated with drug regulatory authority requirements and guidelines associated with one or more jurisdictions. The method comprises the steps of providing, by the processing unit the entity with the regulatory authority guidelines and regulations of the one or more jurisdictions while performing the assigned actions on the dataset at each stage of the project development, and granting access and allowing, by the processing unit, the entity to perform the one or more operations on the dataset at each stage of the project based on the drug regulatory authority guidelines of the corresponding jurisdiction.

In an embodiment, during registration, the method comprises the steps of mapping, through one or more tags, the entity with authentication data comprising any or a combination of a drug generic or proprietary name tag, a product tag, a company tag, a route of administration tag, and a performance tag, and correspondingly generating and storing a set of credentials in a database. Further, when the entity feeds the set of credentials into the system, the processing unit authenticates the entity using the stored set of credentials and correspondingly identifies the access rights, and grants access to the authenticated entity.

In an embodiment, the method comprises the step of allowing, by the processing unit, tracking and monitoring of the one or more projects and the corresponding dataset in real-time; and creating, by the processing unit, one or more reports on the status of the one or more projects and the dataset associated with the corresponding project. In the form of status report, the system provides information on the project that whether the drug substance or product dossier is under process, submitted, under registration or registered

In an embodiment, the method comprises the steps of facilitating, by the processing unit, the administrator to extract parameters of a designated project among the one or more projects, and correspondingly creating and adding one or more templates for a document associated with the project in a database, and allowing the one or more registered entity to access the added template based on the requirement and the access rights assigned to the entity, to enable the preparation of the document.

In an embodiment, the method comprises the steps of enabling, by the processing unit, the approver to create, edit, and delete the project, and create and upload a submission corresponding to a structure of the one or more templates. The processing unit is configured to dynamically provide the drug regulatory authority guidelines and regulations from the database to the approver while performing the actions.

In an embodiment, the method comprises the steps of allowing, by the processing unit, the reviewer to approve or reject the uploaded submission, wherein upon approval, the processing unit uploads the corresponding data on the database.

In an embodiment, in case the reviewer rejects the submission, the processing unit allows the doer to rectify errors in the corresponding data and subsequently allows the doer to upload the updated submission data for the review of the reviewer.

Embodiments of the present invention will be described below in detail with reference to the accompanying drawings.

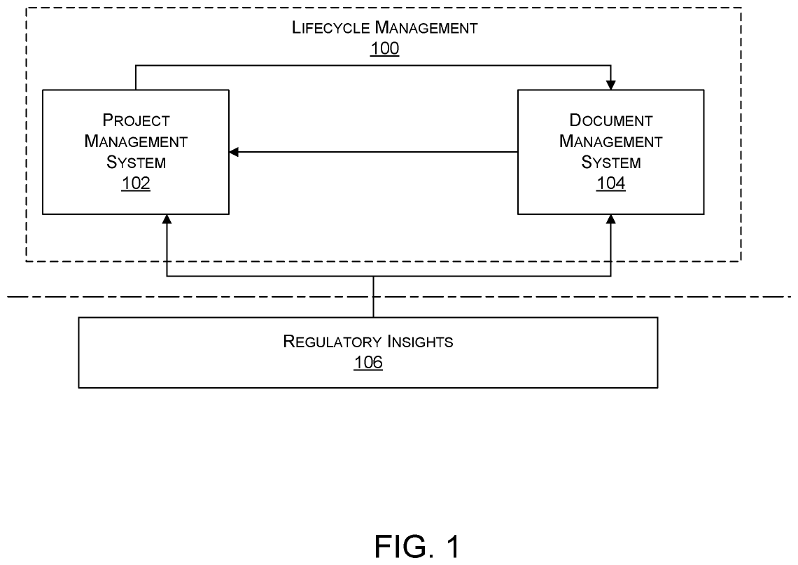

FIG. 1 illustrates a schematic view of a system 100 for project lifecycle management of drug products with regulatory intelligence, according to an embodiment of the present invention. In an embodiment, the system 100 may be implemented in various industries, such as pharmaceutical industry, for managing regulatory information, managing regulatory approval, preparing product submissions, tracking submissions, and monitoring submissions during a lifecycle of a project, such as a drug substance or product including, but not limited to, human and animal drug products comprising of new drugs, generics, new biologics, and biosimilars as well as consumer healthcare products.

During the lifecycle of the medical drug product, from development and manufacturing through to licensing and marketing, at each stage, suitable regulatory information may be required for preparing submissions to ensure the drug has the best chance of approval. In an exemplary implementation, the system 100 may be employed by pharmaceutical companies and agencies to enable them to better understand and manage their product dossiers related to medical drug products for various markets as well as regulatory information and approval lifecycle.

In an embodiment, the system 100 may enable the automation of project workflow by integrating project management 102, document management 104, and regulatory information management 106 with each other. In the project management 102, the system 100 may enable allocation of projects to different resources or entities involved in the project, such as an administrator, an approver, a reviewer, and a doer. The system 100 may allow different resources/entities to perform authorized actions on each of the projects. Further, the system 100 may allow tracking and monitoring of projects in real-time. Furthermore, the system 100 may maintain a complete audit trail of each of the projects created in the system 100.

The system 100 may create a repository of the data used for filing a dossier in different countries. System 100 may allow each of the resources or entities involved in a project to review the earlier filed or approved dossiers in a particular country so as to capture the information based on the learning of review of past dossiers by a regulatory agency. The dossier comprises of several modules such as Administrative Information and Prescribing Information; Common Technical Document Summaries, Quality, Nonclinical Study Reports, and Clinical Study Reports. In an embodiment, in the project management 102, the system 100 may enable the allocation of projects to different resources or entities for each module of the dossier. Further, the system 100 may allow each of the resources/entities to perform various actions on each module and compile the modules into a single dossier after being reviewed by assigned entities.

In the document management 104, the system 100 may allow access to various documents associated with a project to different resources/entities. Further, the system 100 may allow each of the resources/entities to perform various actions, such as reviewing, commenting, approving, rejecting, and authoring, on individual datasets such as documents, folder, files, and the likes associated with the project based on access rights assigned to each of the resources/entities of the project. The system 100 may integrate the document management 104 with the regulatory information management 106 to provide access to data associated with drug regulatory requirements of different countries/regions while performing various actions on individual documents for submission. Further, the system 100 may allow the compilation of the dossier based on documents created by each of the resources/entities related to each module, according to access rights assigned to each of the resources/entities of the project.

The system 100 may be implemented with different software applications executed by a processing unit comprising one or more processors, which may be associated with an electronic device, or a server, or a computing device for performing various actions on the individual datasets associated with the project. In this context, the term “dataset(s)” means any document, or any folder storing multiple documents, or any file storing multiple folders of any format that may be used by a web browser or other applications.

Further, the system 100 may be implemented with different software applications executed by the one or more processors for performing various actions on the individual documents associated with the project. In this context, the term “document(s)” means any document or content of any format including, but not limited to, word processing documents, spreadsheet documents, presentation documents, drawings, webpages, text, etc., that may be used by a web browser or other applications.

Further, the system 100 may allow a resource/entity to maintain complete document status and document audit trail. The system 100 may create reports on the status of various projects and documents associated with such projects. The system 100 may provide a platform to create multiple templates corresponding to the documents associated with different projects. Such templates may be retrieved from a database by a resource/entity based on the requirement of a project to prepare a document associated with the said project. In an embodiment, the database may include, but is not limited to, AWS S3 database and MongoDB database for files and data storage, without departing from the scope of the present invention. In an embodiment, techniques including, but not limited to, Artificial Intelligence (AI) based on Natural Language Processing (NLP) and third-generation Generative Pre-trained Transformer (GPT-3) may be implemented in the database to enhance overall searchability and quality of the data stored in the database.

In the regulatory information management 106, the system 100 may be configured to collect, collate, analyze, and report data associated with drug regulatory authority information of different countries or regions. The system 100 may be configured to manage a database of regulatory information which may be accessed by a resource/entity to retrieve regulatory information relevant to performing an action on a dataset including a document, a folder, and a file associated with a project or a submission. In an embodiment, the database may include, but is not limited to, drug regulatory guidelines and regulations based on country/region, evaluation trends by agencies based on country/region, and queries implemented by different agencies. In an example, the agencies may include, but are not limited to, European Medicines Agency (EMA) and US Food and Drug Administration (FDA). In an implementation, the system 100 may allow the resource/entity to retrieve data associated with at regulatory scenario of a country, Patent scenario, regulatory fee structure, timelines, the approval process of the country, guidelines and templates, waiver situations, and emergency use guidelines, from the database.

Constructional and operational details of the system 100 are explained in the subsequent sections of the present invention.

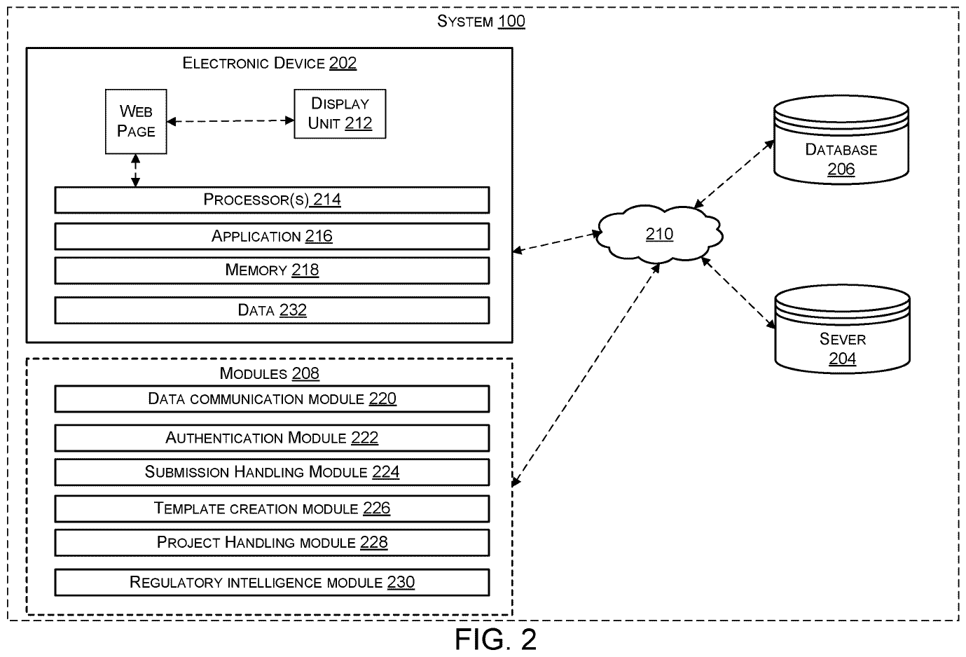

FIG. 2 illustrates a block diagram of the system 100 for project management with regulatory intelligence, according to an embodiment of the present invention. Referring to FIG. 2, in the illustrated embodiment, the system 100 may include, but is not limited to, an electronic device 202, a server 204, a database 206, and modules 208. Each of the electronic device 202, the server 204, the database 206, and the modules 208 may be in communication with each other via a network 210. The network 210 may be a wired network or a wireless network. The network 210 may include, but is not limited to, a mobile network, a broadband network, a Wide Area Network (WAN), a Local Area Network (LAN), and a Personal Area Network. In an embodiment, the electronic device 202 may be embodied as one of a smartphone, a laptop, a tablet, a PDA, and a voice assistant device, without departing from the scope of the present invention.

In an embodiment, the electronic device 202 may include a display unit 212 and a processor 214 communicatively connected to the display unit 212. In an embodiment, the display unit 212 of the electronic device 202 may include, but is not limited to, a touch screen display. Further, the electronic device 202 may also include applications 216 for performing various functionalities. The applications 216 may include, but are not limited to, a spreadsheet application, a document managing application, messaging applications, calendar applications, E-mail applications, and a web-browsing application. In an embodiment, the application 214 may individually be referred to as application 216, without departing from the scope of the present invention.

In an embodiment, the electronic device 202 may include the processor 214, memory 218, and, data 232. The memory 218 is coupled to the processor 214. The processor 214 can be a single processing unit or a number of units, all of which could include multiple computing units. The processor 214 may be implemented as one or more microprocessors, microcomputers, microcontrollers, digital signal processors, central processing units, state machines, logic circuitries, and/or any devices that manipulate signals based on operational instructions. Among other capabilities, the processor 214 is configured to fetch and execute computer-readable instructions and data stored in the memory 218.

The memory 218 may include any non-transitory computer-readable medium known in the art including, for example, volatile memory, such as static random-access memory (SRAM) and dynamic random access memory (DRAM), and/or non-volatile memory, such as read-only memory (ROM), erasable programmable ROM, flash memories, hard disks, optical disks, and magnetic tapes.

The modules 208, amongst other things, include routines, programs, objects, components, data structures, etc., which perform particular tasks or implement data types. The modules 208 may also be implemented as, signal processor(s), state machine(s), logic circuitries, and/or any other device or component that manipulates signals based on operational instructions.

Further, the modules 208 can be implemented in hardware, instructions executed by a processing unit, or a combination thereof. The processing unit can comprise a computer, a processor, such as the processor 214, a state machine, a logic array or any other suitable devices capable of processing instructions. The processing unit can be a general-purpose processor which executes instructions to cause the general-purpose processor to perform the required tasks or, the processing unit can be dedicated to perform the required functions. In another aspect of the present invention, the modules 208 may be machine-readable instructions (software) which, when executed by a processor/processing unit, perform any of the described functionalities.

In an implementation, the modules 208 may include a data communication module 220, an authentication module 222, a submission handling module 224, a template creation module 226, a project handling module 228, and a regulatory intelligence module 230. The data communication module 220, the authentication module 222, the submission handling module 224, the template creation module 226, the project handling module 228, and the regulatory intelligence module 230 are in communication with each other.

The functions of the various elements shown in the figure, including any functional blocks labeled as “processor(s)”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared.

In one embodiment, the modules 208 may be deployed in the server 204 of the system 100 and configured to be in communication with a processor of the server 204. In another embodiment, the modules 208 may be deployed in the electronic device 202 and configured to be in communication with the processor 214 of the electronic device 202. In yet another embodiment, the modules 208 may be deployed in another electronic device 202 and configured to be in communication with the processor 214 of the electronic device 202 and the processor of the server 204.

In one embodiment, the database 206 may be deployed in the server 204 of the system 100. In another embodiment, the database 206 may be deployed separately on a remote server other than the server 204, without departing from the scope of the present invention.

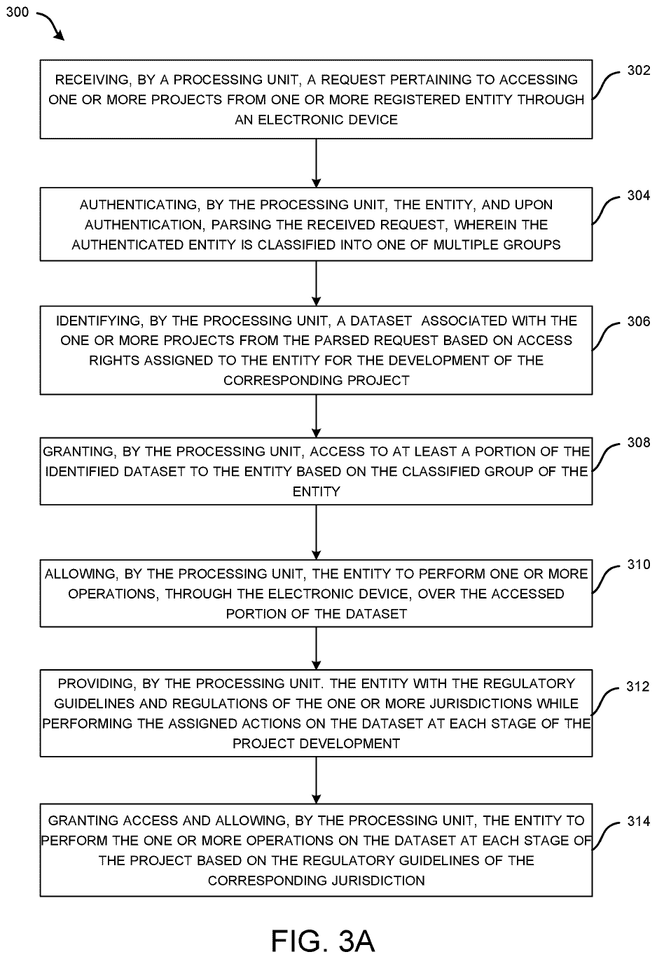

FIG. 3A illustrates a flowchart depicting steps involved in the method 300 for managing the project lifecycle with regulatory intelligence, according to an embodiment of the present invention. Method 300 may involve the hardware elements described and illustrated in FIG. 1 and FIG. 2. For the sake of brevity, details of the present invention that are explained in detail in the description of FIG. 1 and FIG. 2 are not explained in detail in the description of FIG. 3A

In an embodiment, the method 300 may include step 302 of receiving, by a processing unit, a request pertaining to accessing one or more projects from one or more registered entity through an electronic device associated with the registered entity. Method 300 may further include step 304 of authenticating, by the processing unit, the entity that is trying to access the one or more projects at step 304. Further, upon authentication, step 304 may include parsing the received request. The authenticated entity may be classified into one of multiple groups including but not limited to administrator, approver, reviewer, and doer.

Method 300 may further include step 306 of identifying, by the processing unit, a dataset such as a document, folder, and file, associated with the one or more projects from the parsed request based on access rights assigned to the entity for development of the corresponding project. Further, method 300 may include step 308 of granting, by the processing unit, access to at least a portion of the identified dataset to the entity based on the classified group of the entity, followed by step 310 of allowing the entity to perform one or more operations, through the electronic device, over the accessed portion of the dataset. In an embodiment, the one or more operations may include any or a combination of creation, modification, deletion, reviewing, comment, approval, rejection, authoring, and uploading.

Further, method 300 may include step 312 of providing the entity with the drug regulatory guidelines and regulations of the one or more jurisdictions while performing the assigned actions on the dataset at each stage of the project development during step 310. The database associated with the system may be integrated with drug regulatory requirements and guidelines associated with the one or more jurisdictions, such as countries, regions, agencies, and the like. Method 300 may further include step 314 of granting access and allowing the entity to perform the one or more operations on the dataset at each stage of the project based on the drug regulatory guidelines of the corresponding jurisdiction.

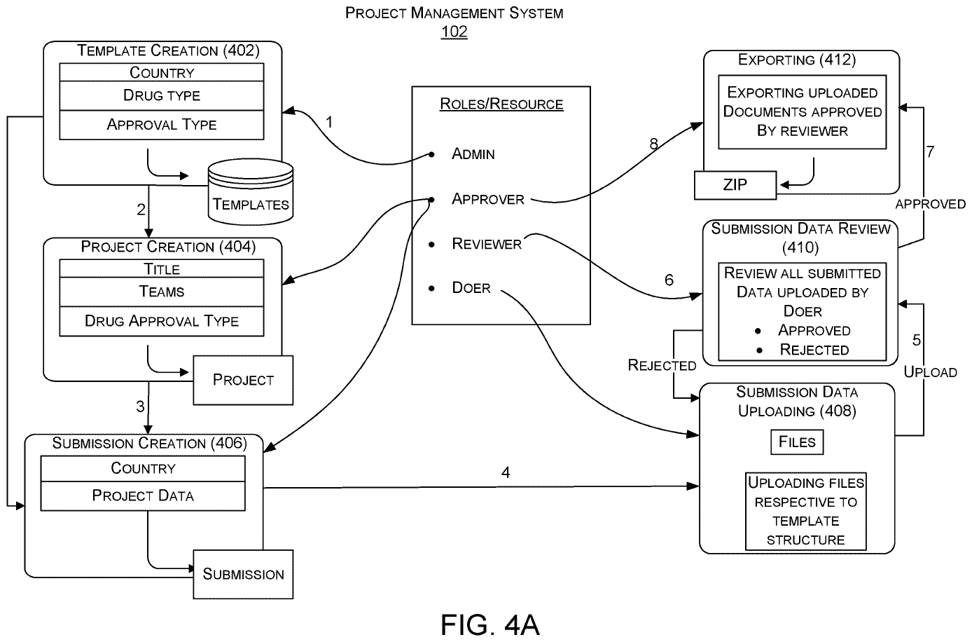

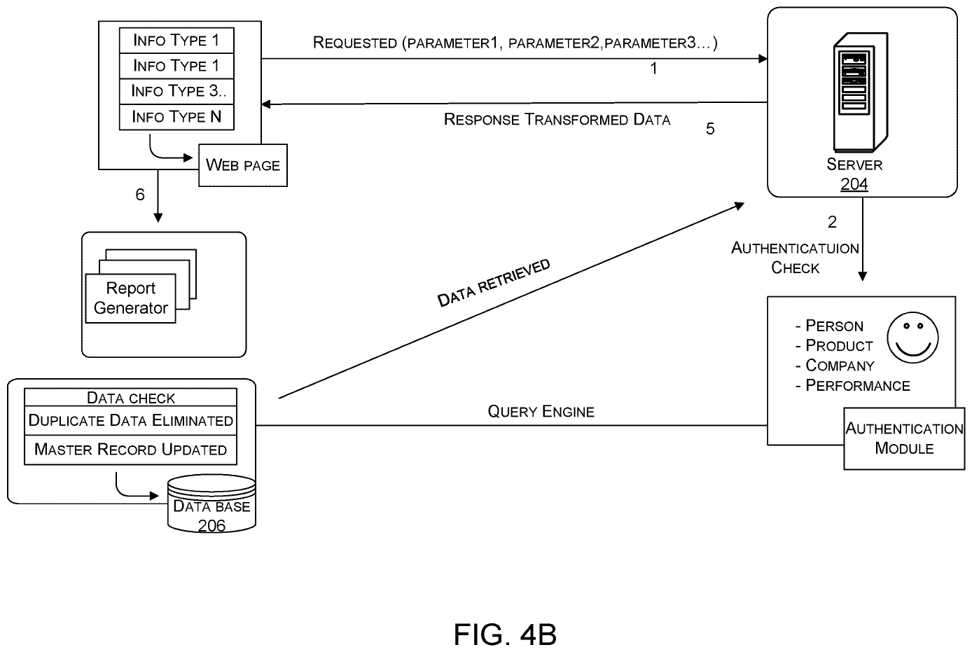

FIG. 4A illustrates a flowchart depicting an operation of the system 100 for managing project lifecycle associated with a medical drug product, according to an embodiment of the present invention. For the sake of brevity, details of the present invention that are explained in detail in the description of FIG. 1 and FIG. 2 are not explained in detail in the description of FIG. 4A. Referring to FIG. 2 and FIG. 4A, the system 100 may be configured to be accessed by a set of users, interchangeably referred to as resources/entities, via a user interface displayed on the display unit 212 of the electronic device 202.

In an implementation, the system 100 may be accessed via a user interface of a webpage displayed on the display unit 212 of the electronic device 202. The processor 214 of the electronic device 202 may execute the web-browsing application to display the user interface of the webpage of the display unit 212 of the electronic device 202. For example, the user interface may be generated as a webpage that is rendered by a web browser executed on the electronic device 202 through the network 210, such as the Internet, or generated by application software (i.e., a mobile app) designed to be executed on a computing device embodied as a wireless communication device, a tablet computer, or other similar types of portable computing device. In an embodiment, the webpage may be created using React.js, Node.js HTML (e.g., HTML5), XML, XHTML, PHP (PHP: Hypertext Pre-processor), JAVASCRIPT™, JAVA™ and/or equivalent programming languages or scripts as generally known in the field of art along with 256-bit encryption for securing the database 206.

The resources/entities may include, but are not limited to, an administrator, an approver, a reviewer, and a doer. Each of the resources/entities may be mapped corresponding to a set of authentication data including, but not limited to, a name tag, a product tag, a company tag, and a performance tag. Information associated with each of the resources/entities may be compared with pre-stored information, stored in the database 206, of the different resources/entities to authenticate the resource/entity and identify the role of such resource/entity in the project. The server 204 may be configured to perform authentication of the resource/entity upon receiving each query from the webpage to determine the role of the resource/entity performing the action on the webpage. Each of the resources/entities may be assigned specific access rights to perform authorized actions on the projects and associated datasets/documents.

Referring to FIG. 4A, at block 402, a resource/entity, such as the administrator (admin), may have access to create, edit, and delete a certain template in the database 206. The administrator may have access to extract the required parameter for adding a new template for a document associated with a project. As shown in FIG. 4A, the administrator may create a template including various fields, such as country, drug type, and approval type. The created template may be accessed by another resource/entity for performing various actions assigned to such resource/entity in the system 100. In an embodiment, data associated with each of the templates may include, but is not limited to, a name of country, a drug type, and a drug approval type. In an embodiment, created templates may be stored in the database 206 of the system 100.

Further, at block 404, a resource/entity, such as the approver, may have access to create, edit, and delete a certain project which will be created based on requirements. In an embodiment, data associated with each of the projects may include, but is not limited to, a title of the project, team structure, and a drug approval type. Upon completion of a project creation, at block 406, the approver may have access to create a submission that is specific to a country or region and associated with a specific project created in the system 100. In an embodiment, the approver may select at least one of the templates stored in the database 206 to create a submission corresponding to a specific structure of such selected template. The submission may be embodied as a document consisting of data associated with the project to be submitted for approval of the product, such as the medical drug product. Further, the system 100 may dynamically provide drug regulatory information from the database 206 to the approver while performing actions, such as create and edit, a certain submission.

In an embodiment, at block 406, a resource/entity, such as the doer, may have access to upload, on the webpage, the created submission for further actions. Further, at block 410, a resource/entity, such as the reviewer, may have access to check or review the uploaded submission from the doer. The reviewer may have access rights to approve the data associated with the submission or reject the data associated with the submission. If the reviewer approves the submission data, then the submission data may be uploaded on the webpage for extraction by another resource/entity, such as the approver. If the reviewer rejects the submission data, then the submission data may be accessed by the doer to rectify any errors in the submission data and subsequently, the doer may upload the updated submission data for the review of the reviewer. Finally, at block 412, the uploaded document approved by the reviewer may be exported by the approver.

FIG. 4B illustrates a flowchart depicting data communication within the system 100 for managing the project lifecycle with regulatory intelligence, according to an embodiment of the present invention. For the sake of brevity, details of the present invention that are explained in detail in the description of FIG. 1, FIG. 2, FIG. 3A, and FIG. 4A are not explained in detail in the description of FIG. 4.

Referring to FIG. 2, FIG. 3A, FIG. 4A, and FIG. 4B, the server 204 may be configured to receive a request indicative of a set of parameters from one of the resources/entities via the user interface of the webpage. The request received by the server 204 may be associated with different actions including, but not limited to, creating projects, creating submissions, editing projects, editing submissions, creating templates, and exporting data associated with submissions and projects. In an embodiment, the server 204 may communicate the received request to the authentication module 222 to identify an assigned role of the resource/entity requesting the set of parameters.

The authentication module 222 may be configured to enable the processing unit to identify the assigned role based on the authentication data mapped corresponding to the respective resource/entity. Upon authenticating the resource/entity, the data communication module 220 may enable the processing unit to communicate the request to the database 206 based on the assigned role of the resource/entity. Based on the request, the data communication module 204 may enable the processing unit to receive data requested by the resource/entity from the database 206. Subsequently, the data communication module 204 may enable the processing unit to export the received data to the server 204. Further, the server 204 may be configured to communicate the exported or transformed data to the webpage displayed on the electronic device 202. Subsequently, the webpage may allow the resource/entity to generate a report based on the exported or transformed data.

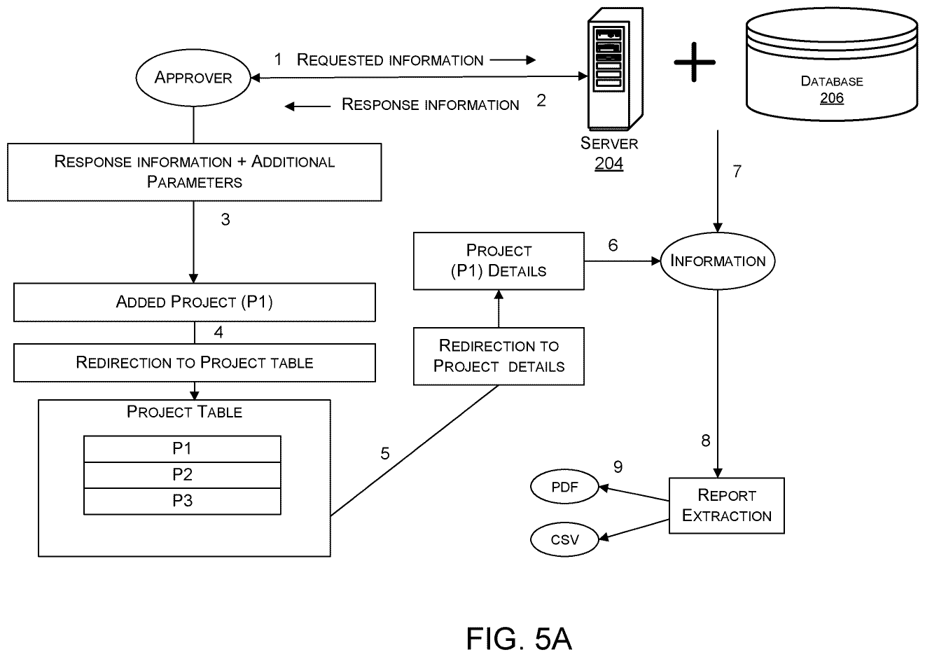

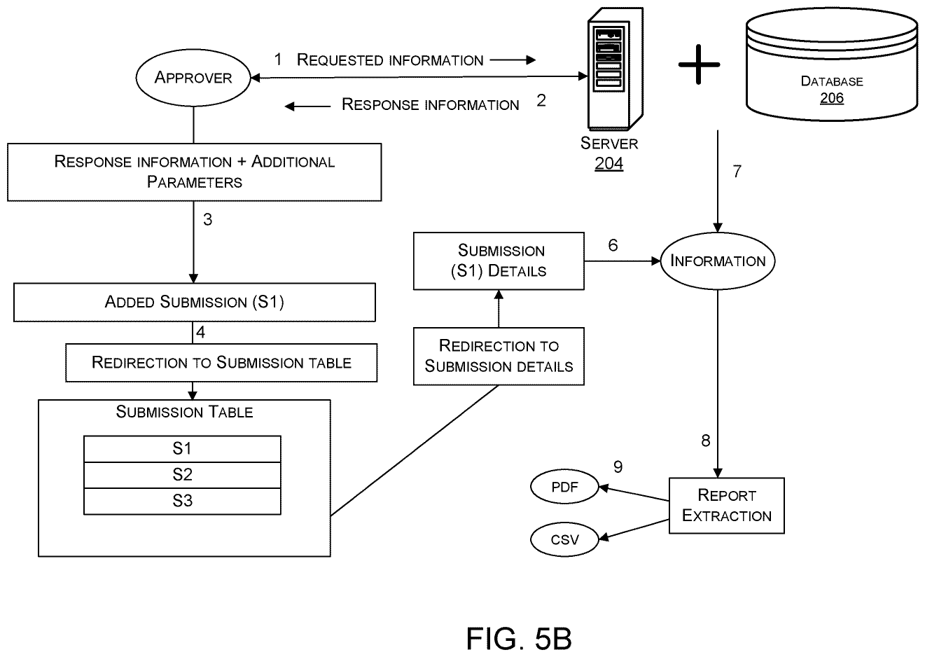

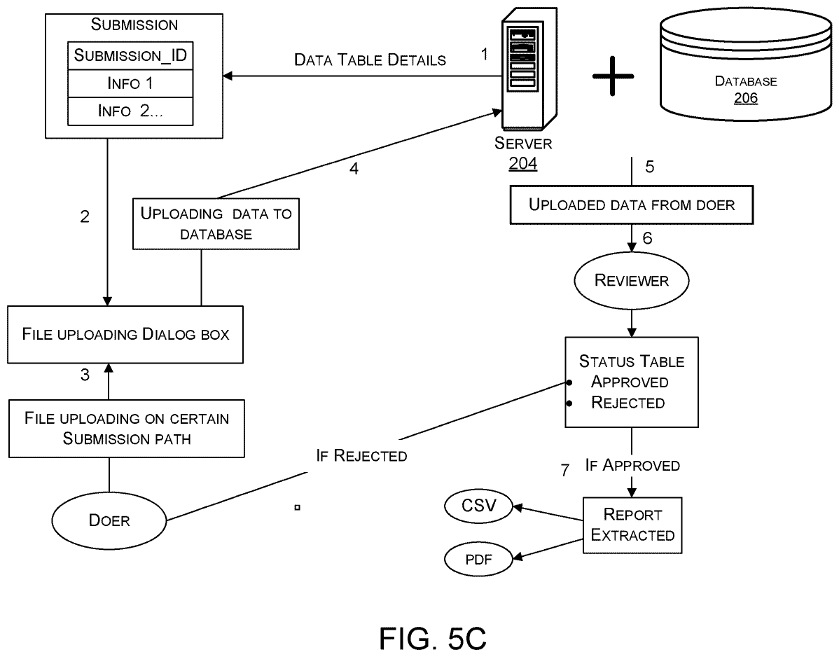

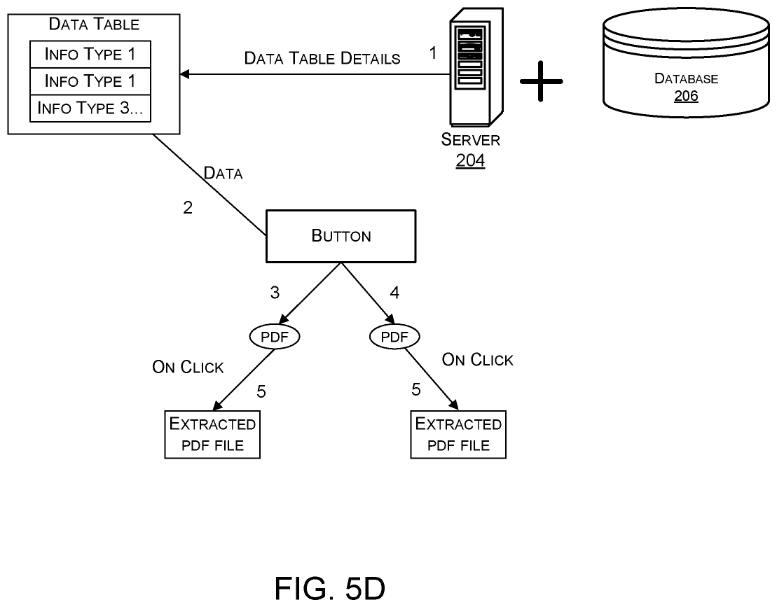

FIG. 5A illustrates a flowchart depicting the creation and extraction of a project by implementing the system 100 for managing the project lifecycle with regulatory intelligence, according to an embodiment of the present invention. For the sake of brevity, details of the present invention that are explained in detail in the description of FIG. 1, FIG. 2, FIG. 3A FIG. 4A, and FIG. 4B are not explained in detail in the description of FIG. 5A.

As explained earlier, the resource/entity, such as the approver, may have access to create, edit, delete, and retrieve a project in the system 100. Referring to FIG. 2, FIG. 3A, FIG. 4A, FIG. 4, and FIG. 5A, the server 204 may be configured to receive a query, from the approver, indicative of a request to retrieve the set of parameters, such as information for adding a new project in the system 100. The server 204 may communicate the query to the project handling module 228. Based on the query, the project handling module 228 may enable the processing unit to retrieve information from the database 206. Subsequently, the data communication module 220 may enable the processing unit to communicate the retrieved information to the server 204. Further, the retrieved information may be communicated to the webpage by the server 204 for the approver. Based on the retrieved information, a new project P1 may be created in the system 100, and data associated with the project P1 may be stored in the database 206.

In an embodiment, upon receiving the retrieved information, the approver may be redirected to another user-interface of the webpage, on the electronic device 202, depicting all added projects P1, P2, P3, . . . Pn in a specific format, such as a tabular format. The webpage may receive a request indicative of a selection of one of the projects, such as P1, from the approver. Based on the selection, the webpage may generate a query indicative of a request to retrieve information associated with the project P1. The electronic device 202 may communicate the generated query from the webpage to the server 204.

Subsequently, the server 204 may be configured to communicate the generated query to the project handling module 228. Based on the query, the project handling module 228 may enable the processing unit to retrieve information associated with the project P1 from the database 206. Subsequently, the data communication module 220 may enable the processing unit to communicate the retrieved information to the server 204. Further, the retrieved information may be communicated to the webpage by the server 204 for the approver. In an embodiment, the approver may have access to extract the retrieved information, from the webpage, associated with one of the projects in a specific format, such as in a spreadsheet.

FIG. 5B illustrates a flowchart depicting the creation and extraction of a submission by implementing the system 100 for managing the project lifecycle with regulatory intelligence, according to an embodiment of the present invention. For the sake of brevity, details of the present invention that are explained in detail in the description of FIG. 1, FIG. 2, FIG. 3A, FIG. 4A, FIG. 4, and FIG. 5A are not explained in detail in the description of FIG. 5B.

As explained earlier, the approver may have access to create a submission that is specific to a country or region. The submission may be referred to as a document associated with a regulatory submission for a medical drug product including drug substance. Each of the projects created in the system 100 may comprise of one or more submissions associated with a regulatory submission for a medical drug product corresponding to different countries or regions. The system 100 may be configured to assign a submission ID identifier to each of the submissions.

Referring to FIG. 2, FIG. 4A, and FIG. 5B, the server 204 may be configured to receive a query, from the approver, indicative of a request to retrieve information for adding a new submission in the system 100 corresponding to a project. The server 204 may communicate the query to the submission handling module 224. Based on the query, the submission handling module 224 may enable the processing unit to retrieve information from the database 206. Further, the submission handling module 224 may enable the processing unit to receive a template from the template creation module 226 based on the query. The submission handling module 224 may also enable the processing unit to receive a set of regulatory information from the regulatory intelligence module based on the query.

Subsequently, based on the set of regulatory information and the received template, the submission handling module 224 may enable the processing unit to generate a structured submission which is communicated to the data communication module 220. Further, the data communication module 220 may enable the processing unit to communicate the structured submission to the server 204. The structured submission may be communicated to the webpage by the server 204 for the approver. The regulatory information may be retrieved from the database 206 dynamically based on data, such as country/region or type of drug approval, associated with the submission. In an embodiment, the regulatory information may be displayed on a user-interactive panel on a user-interface of the webpage displayed on the display unit of the electronic device 202.

In an embodiment, upon receiving the retrieved information, the approver may be redirected to another user-interface of the webpage, on the electronic device 202, depicting all added submissions S1, S2, S3, . . . Pn in a specific format, such as a tabular format. The webpage may receive a request indicative of a selection of one of the submissions, such as S1, from the approver. Based on the selection, the webpage may generate a query indicative of a request to retrieve information associated with the submission S1. The electronic device 202 may communicate the generated query from the webpage to the server 204.

Subsequently, the server 204 may be configured to communicate the generated query to the submission handling module 224. Based on the query, the submission handling module 224 may enable the processing unit to retrieve information associated with the submission S1 from the database 206. Subsequently, the data communication module 220 may enable the processing unit to communicate the retrieved information to the server 204. Further, the retrieved information may be communicated to the webpage by the server 204 for the approver. In an embodiment, the approver may have access to extract the retrieved information, from the webpage, associated with one of the submissions in a specific format, such as in a spreadsheet.

FIG. 5C illustrates a flowchart depicting data uploading associated with a submission by implementing the system 100 for managing project lifecycle with regulatory intelligence, according to an embodiment of the present invention. For the sake of brevity, details of the present invention that are explained in detail in the description of FIG. 1, FIG. 2, FIG. 3A, FIG. 4A, FIG. 4, FIG. 5A, and FIG. 5B are not explained in detail in the description of FIG. 5C.

As explained earlier, the database 206 may be configured to store the templates which are compatible with the requirements of different submissions and drug regulatory requirements of different countries/regions. Referring to FIG. 2, FIG. 3A, FIG. 4A, and FIG. 5C, the resource/entity, such as the doer, may be allowed to select a specific submission created by the approver and stored in the database 206. The webpage may receive an input indicative of a submission ID identifier corresponding to one of the submissions stored in the database 206. Subsequently, the webpage may generate a query indicative of extraction of a template corresponding to such submission along with data associated with the selected submission.

The server 204 may be configured to receive the query from the webpage. Further, the server 204 may communicate the query to the database 206 to extract the template corresponding to the submission along with data associated with the selected submission. The webpage may receive an input from the doer to upload a set of files corresponding to the submission comprising the template and data associated with such submission. In an implementation, upon receiving the template corresponding to the submission along with data associated with the selected submission, a dialog box may be provided on the webpage to allow the doer for uploading the set of files. The webpage may receive an input from the doer indicative of a request to upload the set of files corresponding to the extracted submission. Based on the input, the server 204 may receive data associated with the set of files and subsequently, store the received data in the database 206.

Further, the resource/entity, such as the reviewer, may retrieve data associated with a submission uploaded by the doer. The webpage may receive an input, from the approver, indicative of accessing data, such as the set of files corresponding to a submission, uploaded by the doer. Based on the input, the webpage may generate a query to access the set of files and communicate the query to the server 204. Further, the server 204 may communicate the query to the database 206 and subsequently, the database 206 may communicate the set of files corresponding to the submission to the webpage via the server 204. The reviewer may update a status, such as approved or rejected, corresponding to each of the set of files received from the server 204.

The server 204 may be configured to store data associated with the status corresponding to each of the set of files in the database 206. The resource/entity, such as the doer, may access the status corresponding to each of the set of files. If the status indicates rejection, then the doer may upload an updated set of files as explained earlier in the present invention. If the status indicates approval, then the set of files may be extracted in a specified format from the database 206.

FIG. 5D illustrates a flowchart depicting data exporting associated with a submission or a project by implementing the system 100 for managing project lifecycle with regulatory intelligence, according to an embodiment of the present invention. For the sake of brevity, details of the present invention that are explained in detail in the description of FIG. 1, FIG. 2, FIG. 3A, FIG. 4A, FIG. 4, FIG. 5A, FIG. 5B, and FIG. 5C are not explained in detail in the description of FIG. 5D.

As explained earlier, the resources/entities may have access to extract data, from the webpage, associated with one of the submissions or the projects in a specific format, such as in a spreadsheet or a portable document format. Referring to FIG. 2, FIG. 3A, FIG. 4A, and FIG. 5D, in an embodiment, the webpage may provide a user-selectable button displayed on a user interface of the webpage. The resource/entity may select the user-selectable button for exporting data associated with one of the submissions or the projects in the specific format. Upon receiving an input indicative of the selection, the webpage may provide user-selectable buttons indicating different formats, such as a spreadsheet or a portable document format, for exporting data. Subsequently, based on the selection, the webpage may generate a query to extract the data from the database 206. The server 204 may receive the extracted data in the selected format from the database 206 and subsequently, communicate the extracted data to the webpage.

In an embodiment, the method for project lifecycle management with dynamic integration of regulatory intelligence may be implemented by the system 100 as explained in earlier sections of the present invention.

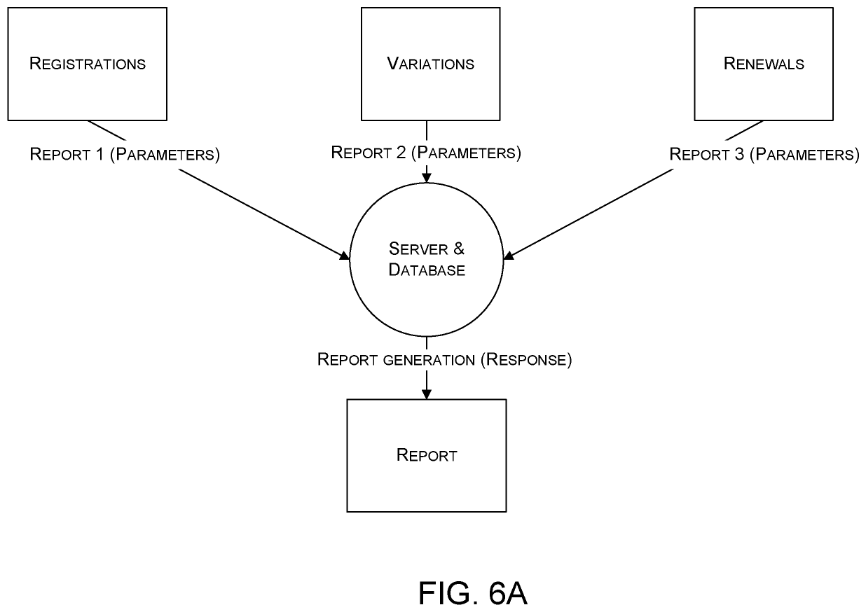

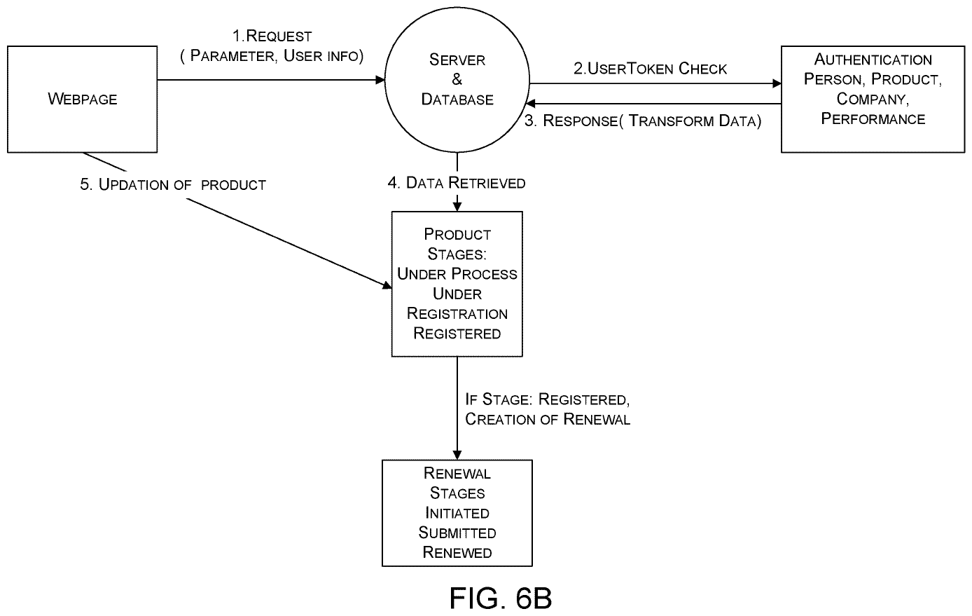

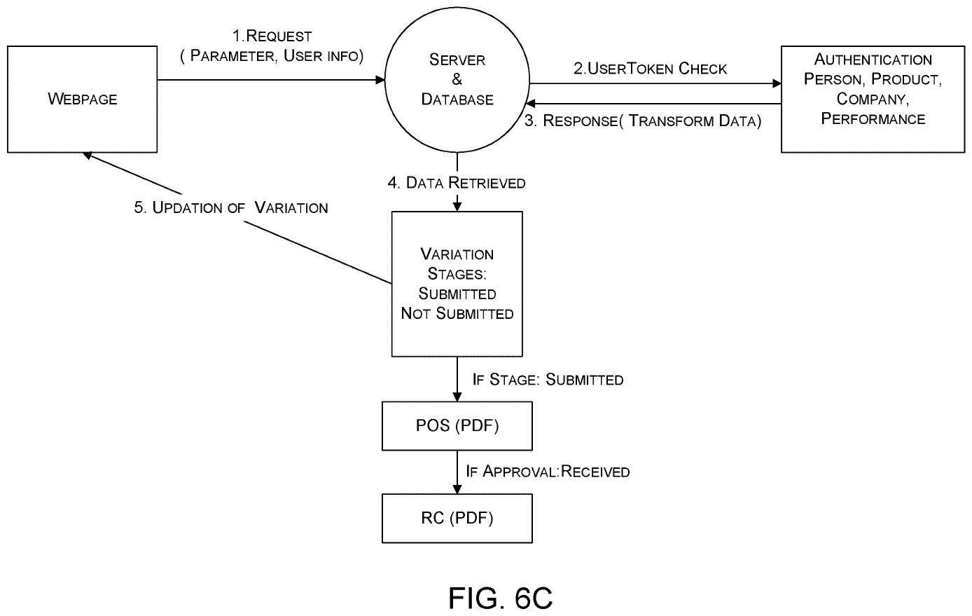

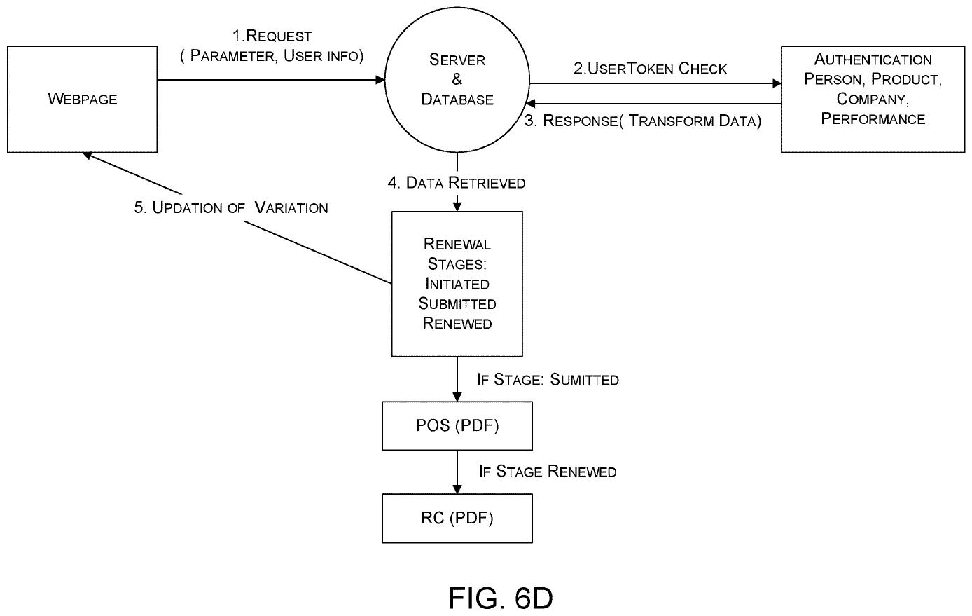

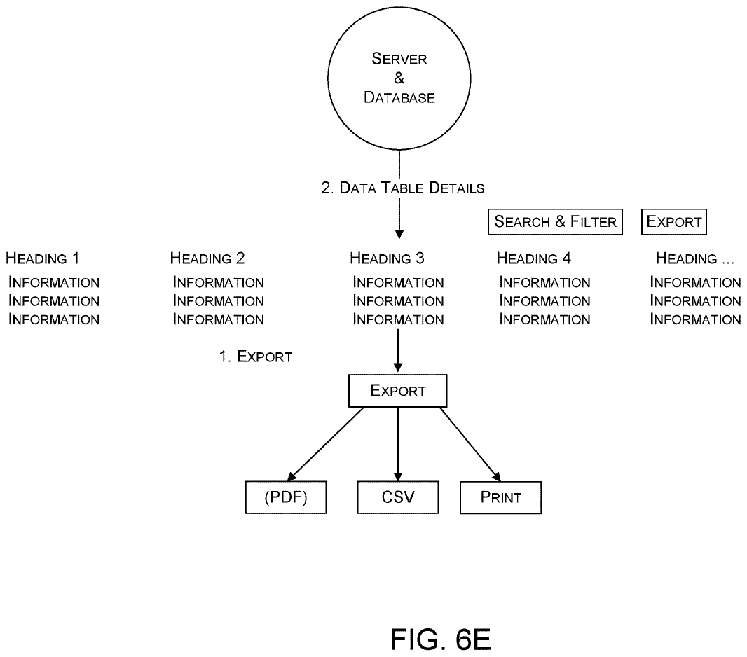

FIG. 6A illustrates a flowchart depicting an overview and working of a regulatory life cycle tracking operation, according to an embodiment of the present disclosure. In an implementation, the system may be employed to track the global regulatory status of filings carried out by an organization, however, other tools and methods may also be employed for regulatory life cycle tracking operations. Those skilled in the art would appreciate that the system is a regulatory lifecycle tracking tool, created for pharmaceutical companies to enable them get ‘At A Glance’ visibility of the global registration status of their products. This system allows the resources/entities to access one or more of the products yet to be filed, a list of products under registration with the agency, variations, renewals, product-wise markets, and market-wise products. This system may track the status of the registration application (submitted/under process/under registration/registered), the status of the variation application (submitted/not submitted/POS status/approved/rejected), and the status of renewal & renewal application (submitted/not submitted/days left to renewal/renewal obtained or not). The detailed operation of the regulatory life cycle tracking operation will be clear in conjunction with FIGS. 6B to 6E below.

Referring to FIG. 6A, data input may be done in three buckets including registration, variation, and renewal. Further, parameters may be set for each bucket and the corresponding information may be transferred to the server and the database. The server may then allow extraction of the stored information in a desired format that may be extracted using the right parameter in the form of a report.

As illustrated in FIG. 6B, registration status may be extractable from the server as per desired parameters (e. g Product, Country, Status, etc). The registration status may be linked to the Renewal bucket.

Further, as illustrated in FIG. 6C, the server may allow resources/entities to extract product-wise and country-wise variation status. Once a variation application is submitted, the Proof of Submission (POS) may be stored in the database which may be retrievable. Likewise, variation applications being authorized/approved by a country agency may also be stored and retrieved as an RC copy.

Furthermore, as illustrated in FIG. 6D, the server allows resources/entities to extract product-wise and country-wise renewal status. Once a renewal application is submitted, the Proof of Submission (POS) may be stored in the database which may be retrievable. Likewise, renewal applications being authorized/approved by a country agency may also be stored and retrieved as an RC copy. Information entered here provides the basis for Renewal alerts. Based on no of days left (from 0 to 270 days, and the like) for renewal, a colour coded alert can be viewed in the dashboard over the electronic device

Referring to FIG. 6E, the server may provide a global report for each module (Registration, Variation, and Renewal), which can be accessed using the electronic device of the resources/entities. The global report may be printable and extractable for each module in the form of PDF as well as CSV downloads. The system allows filtering and customization of the reports as per the criteria requested i.e by product name, country, a route of administration, status, stage, dates, and the like.

### Advantages of the Invention

The above-explained methods and the system 100 for managing project lifecycle with regulatory intelligence leads to at least the following advantages:
Provided an automated platform for reviewing the technical documents pertaining to drug products that are to be registered in a specific country,
Workflow pertaining to review, changes, updates to product dossier, and compilation in Common Technical Document (CTD) format can be managed with ease,
Regulatory projects can be internally tracked and monitored,
Provide dynamic access to regulatory requirements of 130+ countries on the same platform as the document management and submission management, and
Provide access to queries and experiences with country agencies so as to develop a dossier to avoid major queries and thus save time,
Provides a unique combination of project management with document management and regulatory information management system,
In the project management, the system allows project allocation to different resources/entities, multiple levels of project working, allows complete tracking & monitoring of projects on real time-basis, and maintains a complete audit trail,
In the document management, the system allows working on a project related individual documents for reviewing, commenting, authoring, allowing the use of different document management applications, maintains complete document status and document audit trail, allows creating reports on the status of Projects and documents, and allows creating a complete template (e.g. CDT) based database of working documents, and
In the regulatory Information management, the system allows creating a repository of the regulatory information, integrating regulatory information with lifecycle management (project management and document management).

While specific language has been used to describe the present subject matter, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1. A system for regulatory lifecycle management of drug substances or drug products with regulatory intelligence, the system comprising:
a processing unit comprising a processor coupled with a memory, wherein the memory stores one or more instructions executable by the processor to:
receive a request pertaining to accessing the system from one or more registered entity through an electronic device;
authenticate the entity, and upon authentication, parse the received request, wherein the authenticated entity is classified into one of multiple groups; and
identify a dataset associated with one or more projects from the parsed request based on access rights assigned to the entity for development of the corresponding project; and
grant access to at least a portion of the identified dataset to the entity based on the classified group of the entity,
wherein the system allows the entity to perform one or more operations, through the electronic device, over the accessed portion of the dataset, wherein the one or more operations comprise any or a combination of creation, modification, deletion, reviewing, comment, approval, rejection, authoring, and uploading.

2. The system as claimed in claim 1, wherein a database associated with the system is integrated with drug regulatory authority guidelines and regulations associated with one or more jurisdictions, wherein the system is configured to:
provide the entity with the drug regulatory authority guidelines and regulations of the one or more jurisdictions while performing the assigned actions on the dataset at each stage of the project development; and
grant access and allow the entity to perform the one or more operations on the dataset at each stage of the project development based on the regulatory regulations of the corresponding jurisdiction.

3. The system as claimed in claim 2, wherein the group of the entity comprises any of administrator, approver, reviewer, and doer, and wherein the dataset includes any or a combination of a document, a folder, and a file.

4. The system as claimed in claim 3, wherein during registration, the system maps, through one or more tags, the entity with authentication data comprising any or a combination of a generic or proprietary name tag, a product tag, a company tag, a route of administration tag and a performance tag, and correspondingly generates and stores a set of credentials in a database,
wherein when the entity feeds the set of credentials into the system the system authenticates the entity using the stored set of credentials, and correspondingly identifies the access rights, and grants access to the authenticated entity.

5. The system as claimed in claim 3, wherein the system is configured to:
allow tracking and monitoring of the one or more projects and the corresponding dataset in real-time using the electronic device; and
create one or more reports on the status of the one or more projects and the dataset associated with the corresponding project using the electronic device.

6. The system as claimed in claim 3, wherein the system is configured to:
facilitate the administrator to extract parameters of a designated project among the one or more projects, and correspondingly create and add one or more templates for a document associated with the designated project in a database using the electronic device; and
allow the one or more registered entity to access the added template based on the requirement and the access rights assigned to the entity, to enable the preparation of the document using the electronic device.

7. The system as claimed in claim 6, wherein the system enables the approver to create, edit, and delete the project, and create and upload a submission corresponding to a structure of the one or more templates, and wherein the system is configured to dynamically provide the regulatory authority guidelines and regulations from the database to the approver while performing the actions.

8. The system as claimed in claim 7, wherein the reviewer accesses the system, and approves or rejects the uploaded submission, wherein upon approval, the system uploads the corresponding data on the database.

9. The system as claimed in claim 8, wherein in case the reviewer rejects the submission, the system allows the doer to rectify errors in the corresponding data and subsequently allows the doer to upload the updated submission data for the review of the reviewer.

10. The system as claimed in claim 1, wherein the processing unit is associated with any of the electronic device, a central server that is in communication with the electronic device, and a computing device that is in communication with the electronic device and/or the server.

11. The system as claimed in claim 2, wherein the system is configured to allow creation of a repository of the data used in the project for filing a dossier in the one or more jurisdictions, wherein the dossier comprises one or more modules comprising administrative information and prescribing information; common technical document summaries, quality, nonclinical study reports and clinical study reports.

12. The system as claimed in claim 11, wherein the system enables allocation of the projects to the one or more entity for each of the modules of the dossier and allow each of the one or more entity to perform actions on the modules and compile the modules into a single dossier after reviewed by the assigned entities.

13. A method for regulatory lifecycle management of a drug substance or drug product with regulatory intelligence, the method comprising the step of:
receiving, by a processing unit, a request pertaining to accessing one or more projects from one or more registered entity through an electronic device;
authenticating, by the processing unit, the entity, and upon authentication, parsing the received request, wherein the authenticated entity is classified into one of multiple groups;
identifying, by the processing unit, a dataset associated with the one or more projects from the parsed request based on access rights assigned to the entity for the development of the corresponding project;
granting, by the processing unit, access to at least a portion of the identified dataset to the entity based on the classified group of the entity; and
allowing, by the processing unit, the entity to perform one or more operations, through the electronic device, over the accessed portion of the dataset, wherein the one or more operations comprise any or a combination of creation, modification, deletion, reviewing, comment, approval, rejection, authoring, and uploading.

14. The method as claimed in claim 13, wherein a database in communication with the processing unit is integrated with drug regulatory requirements and guidelines associated with one or more jurisdictions, wherein the method comprises the steps of:
providing, by the processing unit. the entity with the drug regulatory authority guidelines and regulations of the one or more jurisdictions while performing the assigned actions on the dataset at each stage of the project development; and
granting access and allowing, by the processing unit, the entity to perform the one or more operations on the dataset at each stage of the project based on the regulatory authority guidelines of the corresponding jurisdiction.

15. The method as claimed in claim 14, wherein the group of the entity comprises any of administrator, approver, reviewer, and doer, and wherein the dataset includes any or a combination of a document, a folder, and a file.

16. The method as claimed in claim 15, wherein during registration, the method comprises the steps of mapping, through one or more tags, the entity with authentication data comprising any or a combination of a generic or proprietary name tag, a product tag, a company tag, a route of administration tag, and a performance tag, and correspondingly generating and storing a set of credentials in a database,
wherein when the entity feeds the set of credentials into the system, the processing unit authenticates the entity using the stored set of credentials and correspondingly identifies the access rights, and grants access to the authenticated entity.

17. The method as claimed in claim 14, wherein the method comprises the step of:
allowing, by the processing unit, tracking and monitoring of the one or more projects and the corresponding dataset in real-time; and
creating, by the processing unit, one or more reports on the status of the one or more projects and the dataset associated with the corresponding project.

18. The method as claimed in claim 14, wherein the method comprises the steps of:
facilitating, by the processing unit, the administrator to extract parameters of a designated project among the one or more projects, and correspondingly creating and adding one or more templates for a document associated with the project in a database; and
allowing, by the processing unit, the one or more registered entity to access the added template based on the requirement and the access rights assigned to the entity, to enable the preparation of the document.

19. The method as claimed in claim 18, wherein the method comprises the steps of enabling, by the processing unit, the approver to create, edit, and delete the project, and create and upload a submission corresponding to a structure of the one or more template, and wherein the processing unit is configured to dynamically provide the regulatory guidelines and regulations from the database to the approver while performing the actions.

20. The method as claimed in claim 19, wherein the method comprises the steps of allowing, by the processing unit, the reviewer to approve or reject the uploaded submission, wherein upon approval, the processing unit uploads the corresponding data on the database.

21. The method as claimed in claim 20, wherein in case the reviewer rejects the submission, the processing unit allows the doer to rectify errors in the corresponding data and subsequently allows the doer to upload the updated submission data for the review of the reviewer.

22. The method as claimed in claim 13, wherein the method comprises the steps of:
generating, by the processing unit, product-wise and country-wise renewal status and renewal alerts based on a number of days left for renewal; and
displaying a color-coded alert for the renewal on a display of the electronic device.
